(19) United States
(12) Patent Application Publication
(10) Pub. No.: US 2006/0282158 A1
(43) Pub. Date: Dec. 14, 2006

(54) Temporary absorbable venous occlusive stent and superficial vein treatment method

(76) Inventor: Syde A. Taheri, Williamsville, NY (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/407,689

(22) Filed: Apr. 20, 2006

Related U.S. Application Data
(63) Continuation of application No. 10/754,919, filed on Jan. 10, 2004.
(60) Provisional application No. 60/520,530, filed on Nov. 17, 2003.

(51) Int. Cl. A61F 2/06

(52) U.S. Cl. 623/1.38

(57) ABSTRACT

A temporary absorbable venous occlusive stent for use in a varicose vein treatment method includes a stent body, a bio-absorbable material associated with the body, and a closure for blocking blood flow past the stent when implanted in a vein. The stent produces localized blood clotting, fibrosis and vein collapse as it is absorbed. A permanent blockage is produced that prevents the undesirable back flow of blood from above the blockage site, thereby reducing distension of a varicose vein below the blockage site.

### CROSS-REFERENCE TO RELATED APPLICATIONS

This application is a continuation of U.S. patent application Ser. No. 10/754,919, filed Jan. 10, 2004, titled TEMPORARY ABSORBABLE VENOUS OCCLUSIVE STENT AND SUPERFICIAL VEIN TREATMENT METHOD, which claims the benefit under 35 U.S.C. § 119(e) of U.S. Provisional Patent Application Ser. No. 60/520,530, filed Nov. 17, 2003, titled TEMPORARY ABSORBABLE STENT. The entire disclosure of each of the above-noted prior application and the above-noted provisional application is hereby incorporated by reference herein and made a part of this specification.

### BACKGROUND OF THE INVENTION

1. Field of the Invention

This invention relates to vascular disease and the treatment thereof. More particularly, the invention pertains to an apparatus and method for treating varicose veins.

2. Description of the Prior Art

By way of background, the most common technique for treating varicose veins is to remove one or more superficial blood vessels by way of surgical ligation and stripping. This technique suffers from the usual drawbacks associated with invasive procedures, i.e., the need for hospitalization and surgery under general anesthesia, associated pain, infection risk, prolonged recovery time, limited mobility during recovery, permanent scarring, etc. More recently, an obliteration technique using high temperature radio frequency ablation has been proposed. According to this technique, a section of superficial vein is ablated so as to produce a blockage that prevents excessive blood flow from above the treatment site, thereby reducing vein distension below the site. The radio frequency ablation technique represents an improvement over surgical ligation and stripping because it can be performed percutaneously as an ambulatory procedure. However, there is a risk that the high radio frequency energy could damage tissue that surrounds the treated area if the procedure is not performed properly. Moreover, the electronic equipment and procedural instrumentation required for the radio frequency technique are relatively costly. There is also no way to readily confirm that the treatment has been effective without the use of a venogram requiring dye injection and X-ray imaging.

It would be desirable if varicose veins could be treated in a manner that overcomes the foregoing disadvantages of existing techniques.

### SUMMARY OF THE INVENTION

The foregoing problems are solved and an advance in the art is obtained by a novel temporary absorbable venous occlusive stent and a related varicose vein treatment method. The stent includes a stent body, a bio-absorbable material associated with the body, and a closure for blocking blood flow past the stent when implanted in a vein. The stent promotes localized blood clotting, fibrosis and vein collapse as the stent is absorbed. A permanent blockage is thereby produced that prevents the undesirable back flow of blood from above the stent implantation site, thereby reducing distension of the varicose vein below the implantation site.

According to the inventive treatment method, a temporary absorbable venous occlusive stent is introduced via a deep venous system or superficial venous system approach to an implantation site proximate to or above a varicose vein to be treated. There, the stent is deployed against the walls of the vein. Closure of the stent is performed as necessary to block blood flow past the stent. As indicated above, the stent is gradually absorbed while producing a permanent blockage resulting from localized blood clotting, fibrosis and vein collapse.

### BRIEF DESCRIPTION OF THE DRAWINGS

The foregoing and other features and advantages of the invention will be apparent from the following more particular description of preferred embodiments of the invention, as illustrated in the accompanying Drawings, in which:

FIG. 1 is a diagrammatic view showing the outline of a human upper thigh and groin area and a portion of the venous circulatory system therein;

FIG. 2 is a perspective view showing a temporary absorbable venous occlusive stent in accordance with a first exemplary embodiment of the invention in various stages of closure;

FIG. 3A is a longitudinal cross-sectional view of a varicose vein and a perspective view showing the stent of FIG. 2 being inserted therein;

FIG. 3B is a view according to FIG. 3A showing the stent in an initial deployed condition in the varicose vein;

FIG. 3C is a view according to FIG. 3A showing the stent in a final deployed condition in the varicose vein;

FIG. 3D is a view according to FIG. 3A showing the stent in a partially absorbed condition and the varicose vein in a state of partial collapse;

FIG. 3E is a view according to FIG. 3A after the stent has been completely absorbed and the varicose vein is fully collapsed and permanently blocked;

FIG. 4A is a perspective view of a temporary absorbable venous occlusive stent in accordance with a second exemplary embodiment of the invention in which the stent is formed as a generally tubular member having a closed end;

FIG. 4B is a perspective view according to FIG. 4A in which the stent is cross-sectionally divided to illustrate its hollow interior;

FIG. 5A is a perspective view of a temporary absorbable venous occlusive stent in accordance with a third exemplary embodiment of the invention in which the stent is formed as a solid member;

FIG. 5B is a perspective view according to FIG. 5A in which the stent is cross-sectionally divided to illustrate its solid interior;

FIG. 6A is a longitudinal cross-sectional view showing a portion of a venous circulatory system in a human upper thigh and groin area and a perspective view of a stent delivery system including a guide wire that has been advanced from a percutaneous point of entry (not shown) to the site of a varicose vein to be implanted, and a sheath introducer passing over the guide wire and approaching the sapheno-femoral junction;

FIG. 6B is a view according to FIG. 6A showing the sheath introducer after it has been advanced through the long saphenous vein (and any intervening superficial branch veins) to the site of the varicose vein to be implanted;

FIG. 6C is a view according to FIG. 6A showing a balloon catheter carrying a temporary absorbable venous occlusive stent within the sheath introducer, with the stent approaching the sapheno-femoral junction;

FIG. 6D is a view according to FIG. 6A showing the stent at the distal end of the sheath introducer;

FIG. 6E is a view according to FIG. 6A showing the stent following deployment in the varicose vein to be implanted and expansion by the balloon catheter, and further showing withdrawal of the balloon catheter back into the sheath introducer;

FIG. 6F is a view according to FIG. 6A following removal of the balloon catheter from the sheath introducer;

FIG. 6G is a view according to FIG. 6A showing the closure of the stent using drawstring members extending from the stent to the percutaneous entry point;

FIG. 6H is a view according to FIG. 6A following securement of the stent drawstring members and cutting thereof proximate to the stent;

FIG. 6I is a view according to FIG. 6A following removal of the sheath introducer;

FIG. 6J is a view according to FIG. 6A showing the stent in a partially absorbed condition and the varicose vein in a state of partial collapse;

FIG. 6K is a view according to FIG. 6A after the stent has been fully absorbed and the implanted section of varicose vein is fully collapsed and permanently blocked;

FIG. 7 is a longitudinal cross-sectional view showing a portion of a venous circulatory system in a human upper thigh and groin area and a perspective view of a stent delivery system including a guide wire having a ferromagnetic tip that has been advanced at least partially under the guidance of a magnet from a percutaneous point of entry (not shown) to the site of a varicose vein to be implanted, and a sheath introducer passing over the guide wire and approaching the sapheno-femoral junction; and

FIG. 8 is a longitudinal cross-sectional view showing a portion of a venous circulatory system in a human upper thigh and groin area and a perspective view of a stent delivery system including an sheath introducer having a ferromagnetic tip that has been guided at least partially by way of a magnet to the sapheno-femoral junction.

### DETAILED DESCRIPTION OF EXEMPLARY EMBODIMENTS

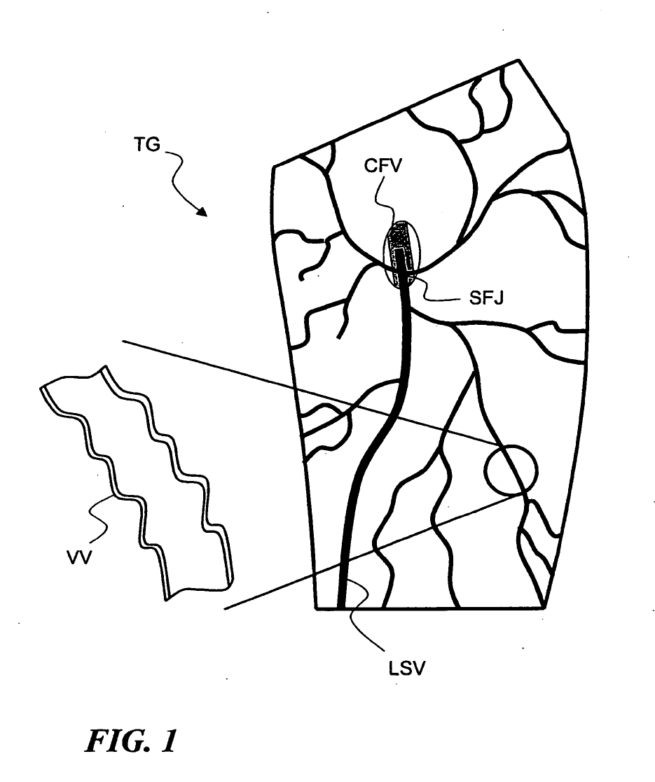

Turning now to FIG. 1, the thigh-groin region (TG) of a human leg is shown to illustrate a portion of the venous circulatory system and an exemplary area of the human body in which the present invention may be implemented. In particular, FIG. 1 shows the sapheno-femoral junction (SFJ) where many of the superficial leg veins come together before joining the common femoral vein (CFV). Although not shown, the flow of blood at the sapheno-femoral junction is controlled by a one-way valve that is designed to direct blood inwardly and upwardly, helping it return toward the heart. If this valve fails to function properly, some blood is able to flow back down the leg, increasing the pressure in the superficial veins and their branches. The long saphenous vein (LSV) is one of the main superficial veins in the thigh. If extra blood is forced into this vein by a leaking valve at the sapheno-femoral junction, the vein stretches and further valves within it become distorted and begin to leak. Blood is then able to flow further down the leg in the wrong direction, eventually filling and distending more and more branches, causing the appearance of varicose veins such as the varicose vein (VV) shown in the inset in FIG. 1. Similar problems can occur due to valve leakage at the sapheno-popliteal junction of the short saphenous vein and the popliteal vein behind the knee. Leakage of any of the valves in the perforator veins connecting the superficial leg veins to the deep veins of the leg can likewise lead to varicose veins.

The present invention contemplates a varicose vein treatment apparatus and method wherein a distended varicose vein, such as the vein VV of FIG. 1 is implanted with a temporary absorbable venous occlusive stent. The stent is placed either within the distended area or into an adjacent (or non-adjacent) venous section that is delivering unwanted downward blood flow to the distended area. The stent stops the flow of blood past the implantation site and promotes localized blood clotting, fibrosis and vein collapse as the stent is absorbed. Following complete absorption of the stent, a permanent blockage remains that prevents blood flow from above, thereby reducing or eliminating vein distention below the implantation site.

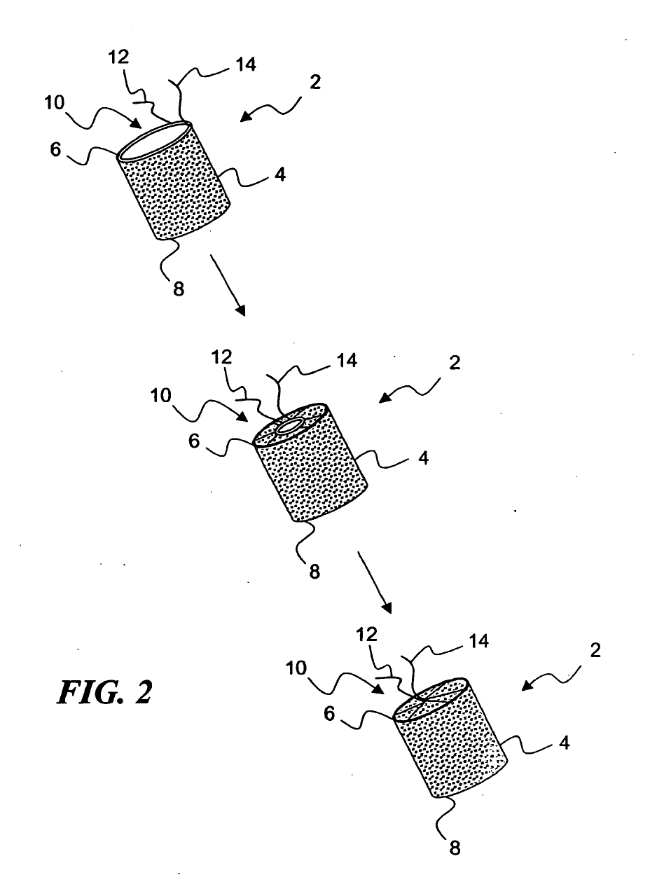

FIG. 2 illustrates an exemplary embodiment 2 of a temporary absorbable venous occlusive stent that may be used in accordance with the invention. The stent 2 is configured as a generally tubular body 4 having a proximal end 6 and a distal end 8. The body 4 is made from a bio-absorbable material having the capability to absorb within a time frame that is long enough to allow the aforementioned permanent blockage to form in a vein to be implanted. By way of example only, a fabric woven from threads of dissolvable (e.g., polylactic acid) suture material could be used to form the body 4. Such material has an absorption schedule of about 28 days, which should be more than adequate for purposes of the present invention. Both ends of the stent 2 are initially open, but the proximal end 6 is provided with a suitable closure system that allows it to be closed following deployment.

FIG. 2 shows one exemplary closure system in the form of a drawstring arrangement. In particular, a drawstring 10 made from a dissolvable suture or other bio-absorbable material is secured around the circumferential periphery of the proximal end 6 of the body 4 in a manner that allows the proximal end to be closed by pulling on the drawstring's end portions 12 and 14. Although not shown, the drawstring 10 could likewise be placed at the distal end 8 of the stent 2. It could also be arranged on the stent 2 so that only a single drawstring end portion is required for stent closure.

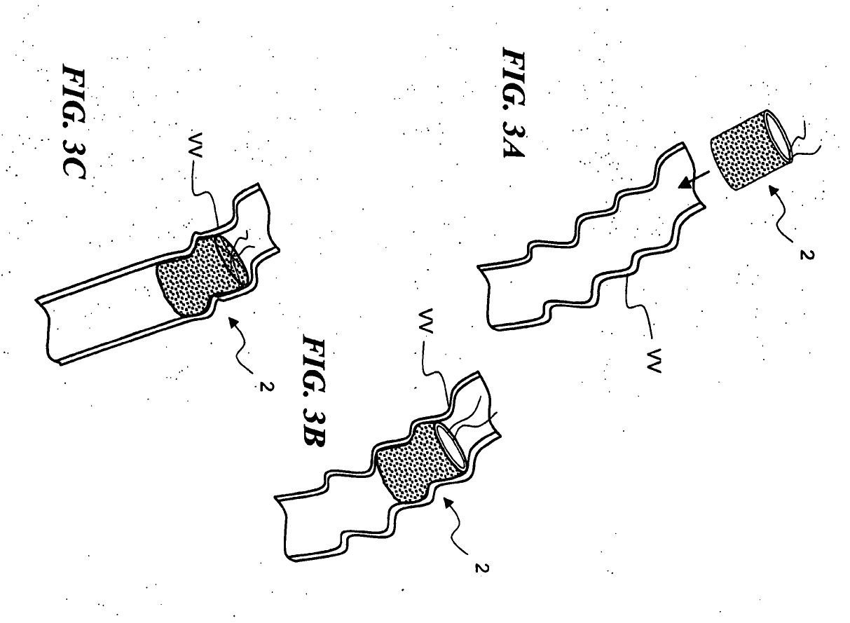

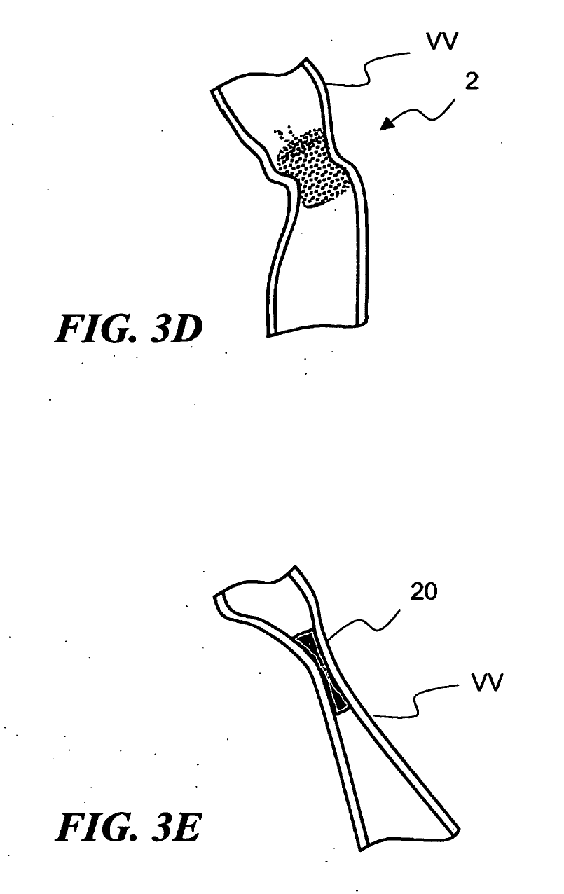

FIGS. 3A-3E illustrate a series of the sequential steps by which the stent 2 can be used to treat a varicose vein. In FIG. 3A, the stent 2 is in the process of being deployed to the varicose vein (VV) to be treated, with the stent's proximal end being oriented toward its point of entry into the patient (not shown). In FIG. 3B, the stent 2 is shown at an implantation site within the vein. In FIG. 3C, the drawstring 10 has been manipulated to close the stent's proximal end 6. At this point, blood flow is prevented from passing through the stent 2, such that distention of the vein should be alleviated. Closure of the stent 2 also causes blood in the vicinity of the stent to pool and begin clotting. This produces fibrotic tissue and vein collapse as the stent is absorbed. FIG. 3D shows the stent 2 in a partially absorbed condition and the vein in a state of partial collapse. In FIG. 3E, the stent 2 is completely absorbed and the vein is fully collapsed and permanently blocked at 20. The blockage 20 prevents the back flow of blood from leaky vein valves situated above the implantation site.

It will be appreciated that many alternative constructions may be used to provide a temporary absorbable venous occlusive stent in accordance with the invention. For example, such stents may be produced in a variety of diameters and lengths for implantation at different locations of the body. It may also be desirable to utilize several stents at a single implantation site in lieu of a single longer stent. In that case, the several stents could be provided with suitable connectors for establishing serial interconnections between adjacent stents. Stents in accordance with the invention can also be treated with a suitable drug, such as rapamycin (a cell cycle inhibitor). Such drug-alluded stents may be more efficient promoters of vein collapse than untreated stents. Another alternative would be to treat a stent with a suitable radioactive substance that produces localized cell death and an increased rate of vein collapse. Stents in accordance with the invention could be treated with drugs or radioactive substances by via impregnation into the bio-absorbable material that forms the stents. Alternatively, the stents could be formed with a double lumen or the like to provide an enclosed pocket for containing a drug or radioactive substance. Such a pocket could also be used to carry a cryomaterial that further promotes cell death and vein collapse. Another use for a pocket formed on the stent would be to carry a dye material to guide stent placement at an implantation site. Implantation could also be aided by providing the stent with a radioopaque marker.

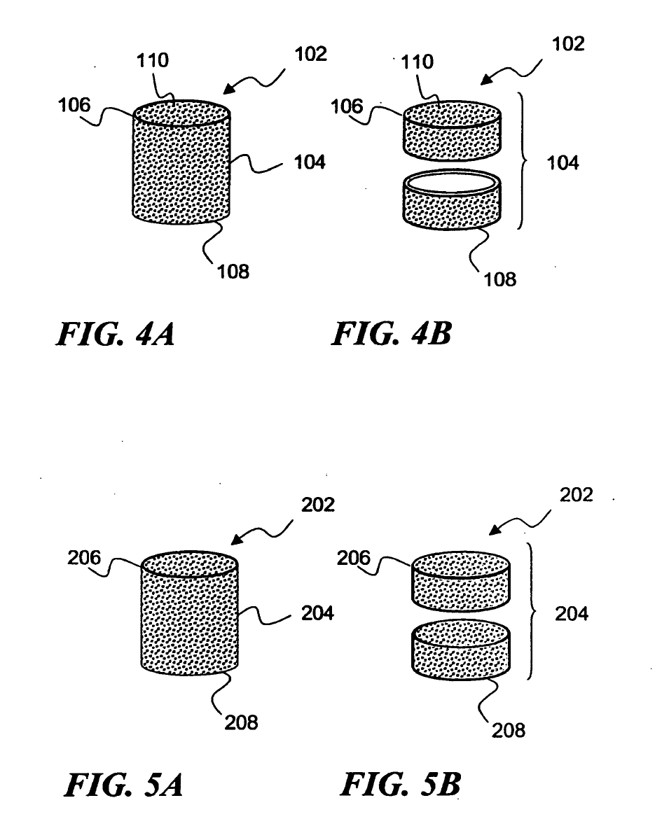

FIGS. 4A-4B and 5A-5B illustrate additional stent configuration alternatives. In FIG. 4A, a second exemplary embodiment 102 of a temporary absorbable venous occlusive stent is shown. As can be seen with additional reference to FIG. 4B, the stent 102 is configured as a generally tubular body 104 having a proximal end 106 and a distal end 108. The body 104 is similar to the body 4 of FIG. 2 except that the proximal end 106 of the body 104 has a closed end wall 110. The end wall 110 provides a closure system for the stent 102 that represents an alternative to the drawstring closure system used in the stent 2 of FIG. 2. Note that the end wall 110 can either be permanently formed as part of the body 104, or alternatively could be separately attached thereto, either prior to, during or after deployment of the stent 102 (e.g., as an insertable plug). Although the end wall 110 is located at the proximal end 106 of the stent 102, it could also be located at the distal end 108. A wall could also be located at any point between the ends 106 and 108 of the stent 102, such as at the stent's longitudinal midpoint.

In FIG. 5A, a third exemplary embodiment 202 of a temporary absorbable venous occlusive stent is shown. As can be seen with additional reference to FIG. 5B, the stent 202 is configured as a generally solid cylindrical body 204 having a proximal end 206 and a distal end 208. The use of a solid body 204 provides a closure system for the stent 202 that represents an alternative to the drawstring closure system used in the stent 2 of FIG. 2. The body 204 can be made from any suitable bio-absorbable material, such as packed or bundled bio-absorbable filaments, folded bio-absorbable fabric, or a bio-absorbable foam. Although the body 204 is shown as being generally cylindrical, it will appreciated that other configurations could also be used, such as spheres, cones, pyramids, irregular shapes, etc., to implement a body portion of the stent 202.

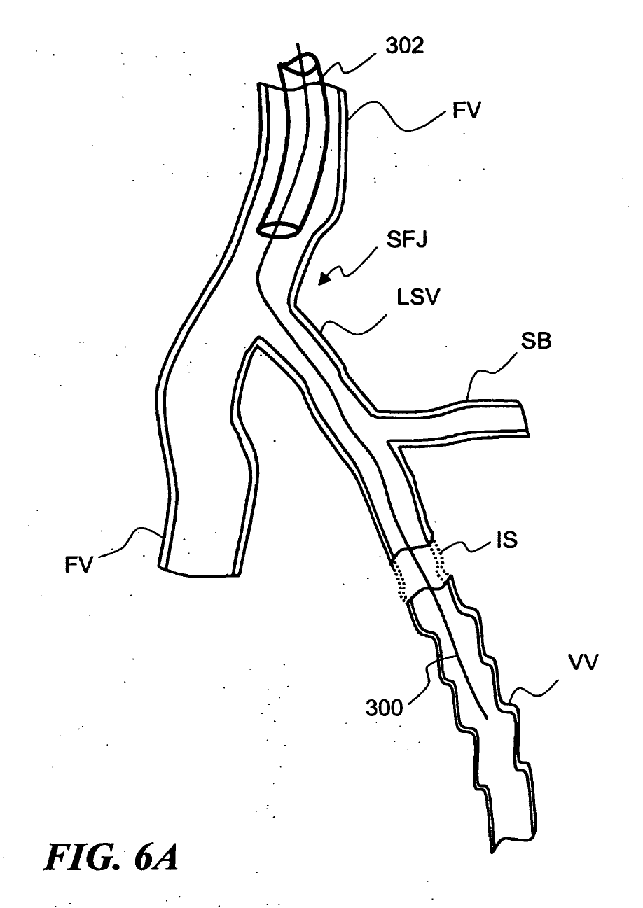

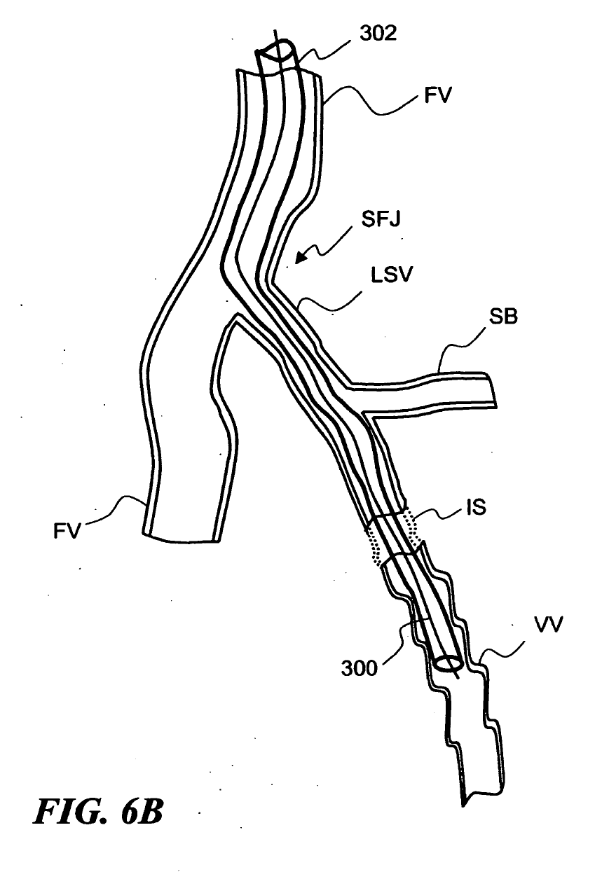

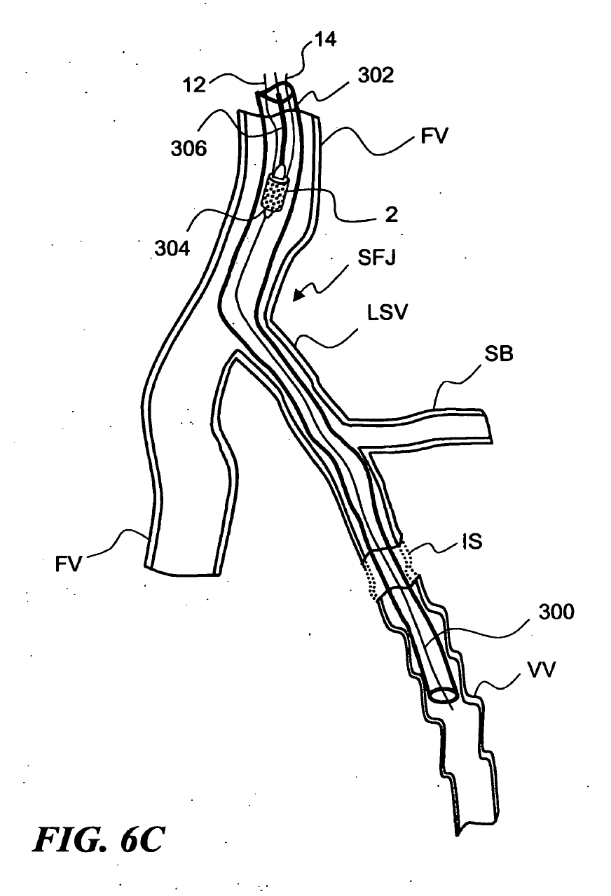

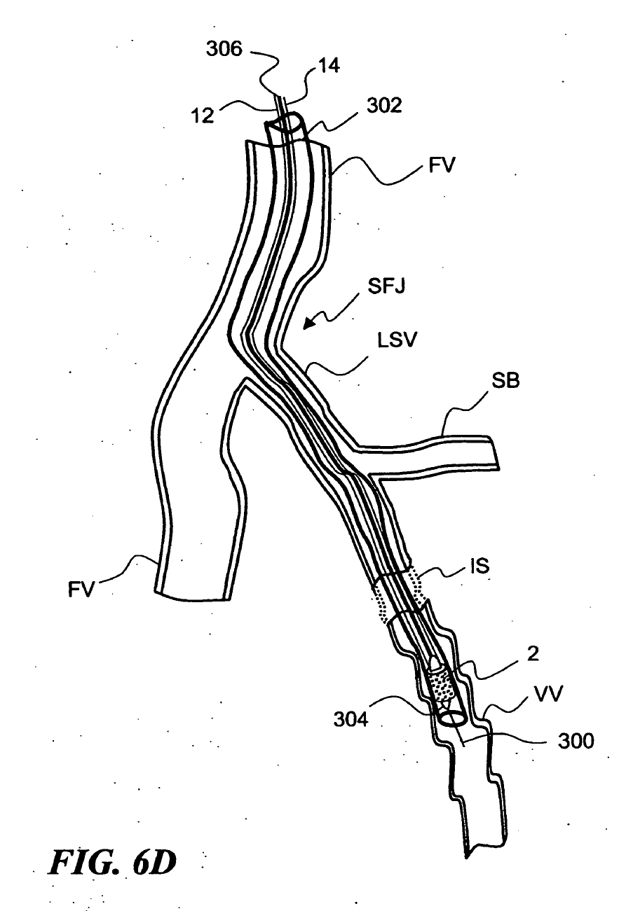

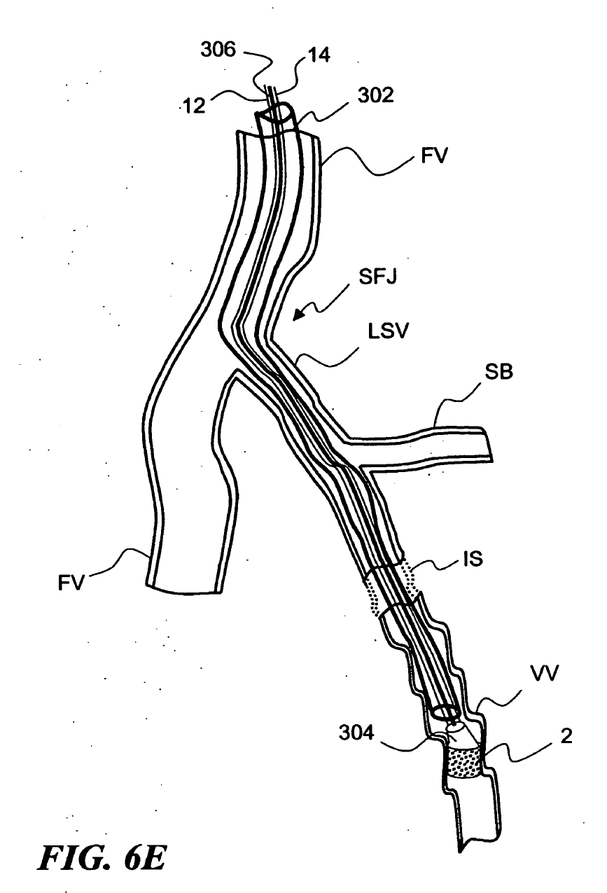

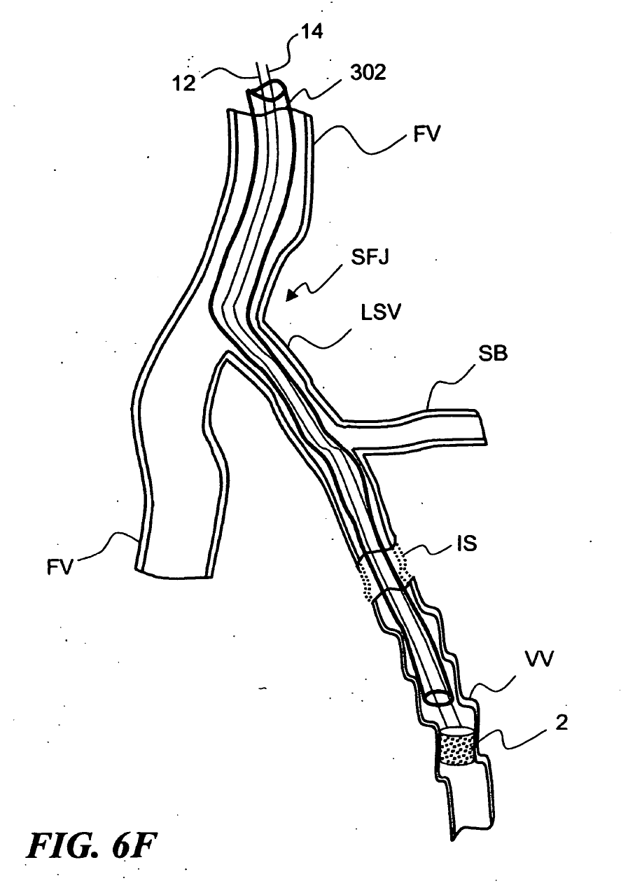

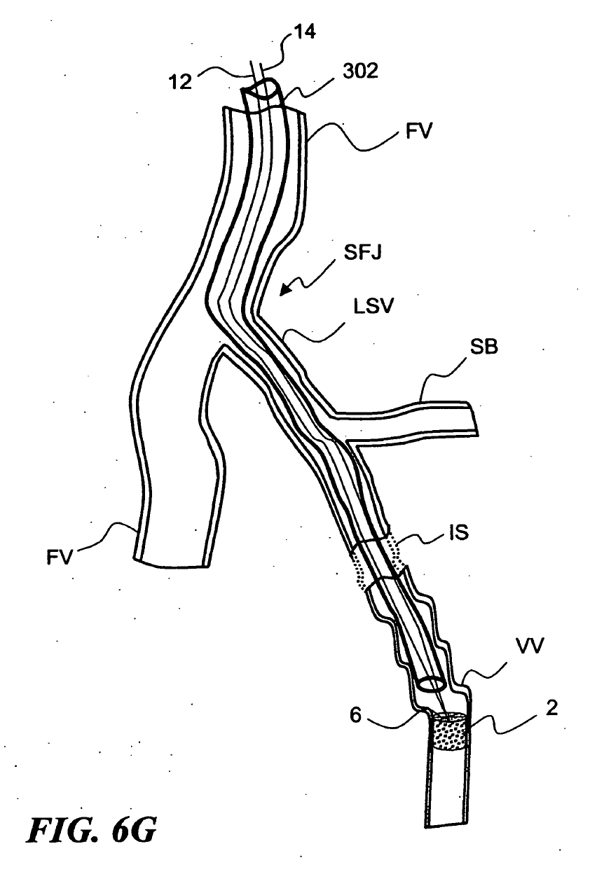

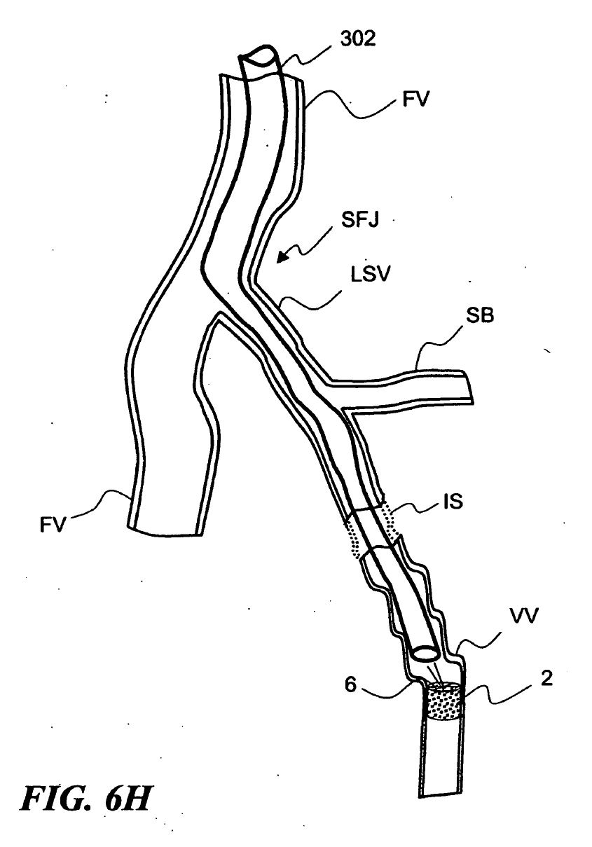

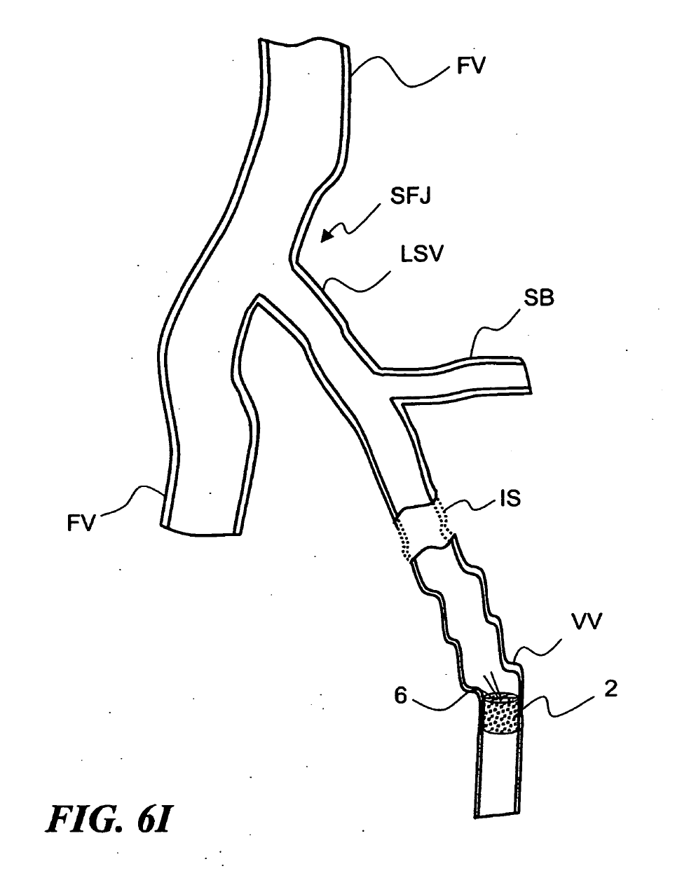

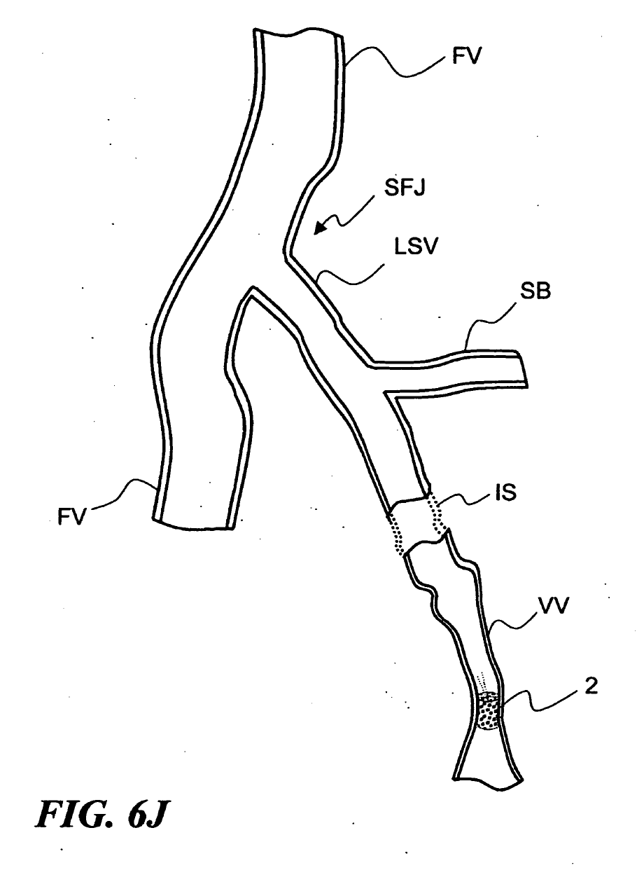

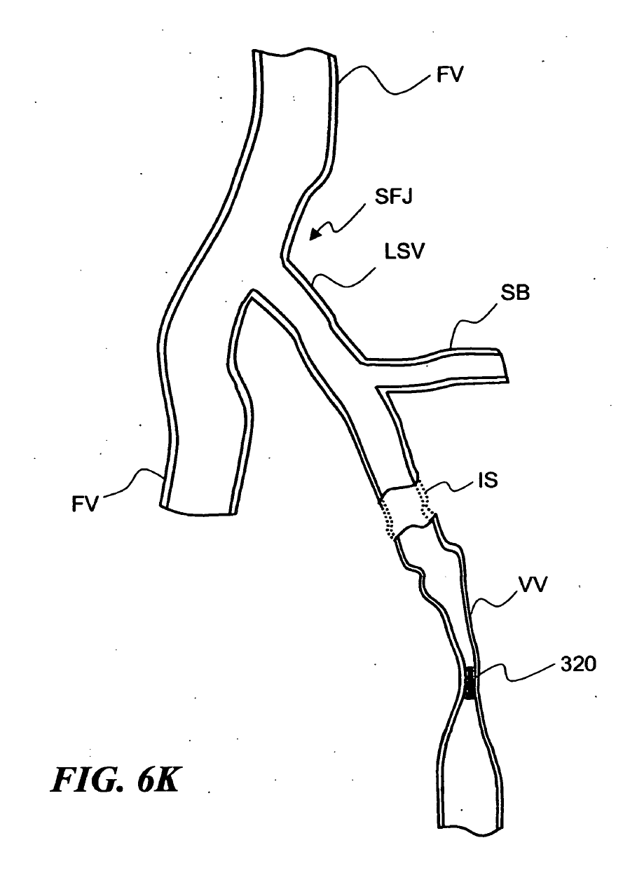

Turning now to FIGS. 6A-6K, an exemplary stent implantation method utilizing pathways within a patient's deep vein system will now be described. It is assumed for the purpose of illustration only that the temporary absorbable venous occlusive stent 2 of FIG. 2 is to be implanted in a varicose vein (VV) in one of the patient's legs. It is further assumed that this vein can be reached via a common femoral vein (FV), a sapheno-femoral junction (SFJ), a long saphenous vein (LSV), and a possible intervening section (IS) that may contain one or more side branches of the type shown by reference numeral (SB). According to the exemplary method, a percutaneous opening (not shown) is formed in the patient's cephalic vein located in the upper arm (or any other suitable location that allows access to the patient's deep venous system). Following vein entry, an optional guide wire is introduced and passed upwardly (e.g., using conventional optical guidance means as necessary) through the subclavian vein, then downwardly through the superior vena cava and the inferior vena cava to a desired one of the femoral veins. Advancement of the guide wire then continues along the selected femoral vein to the sapheno-femoral junction, at which point the long saphenous vein is entered. The guide wire is then further advanced along appropriate tributaries of the long saphenous vein until the implantation site in the varicose vein (VV) is reached. As shown in FIGS. 6A and 6B, a sheath introducer is introduced over the guide wire and advanced along the venous pathways in which the guide wire is situated until the distal end of the introducer is adjacent to the implantation site. Alternatively, the sheath introducer can be inserted without the use of a guide wire, or a guide wire could be inserted after the sheath introducer (in order to guide a balloon catheter as described below). In FIGS. 6A and 6B, the guide wire is designated by reference numeral 300 and the sheath introducer is designated by reference numeral 302.

Turning now to FIGS. 6C and 6D, the stent 2 of FIG. 2 is mounted on an inflatable balloon dilator 304 situated at the end of a balloon catheter 306, and the catheter is advanced over the guide wire 300 (if present) to the distal end of the sheath introducer 302. Alternatively, the balloon dilator 304 and the stent 2 could be positioned at the distal end of the sheath introducer 302 prior to the latter's introduction into the patient, such that the stent is carried with the sheath introducer to the implantation site. As can be further seen in FIGS. 6C and 6D, the stent's drawstring ends 12 and 14 will extend back to the percutaneous entry site (not shown) as the stent 2 is advanced into the patient.

In FIG. 6E, the stent 2 has been deployed out of the sheath introducer 302 by advancing the balloon catheter 306, the balloon dilator 304 has been dilated to expand the stent against the vein walls, and the balloon catheter is in the process of being removed from the stent. In FIG. 6F, the balloon catheter 306 has been removed from the introducer catheter 302. In FIG. 6G, the drawstring ends 12 and 14 have been manipulated to close the proximal end 6 of the stent 2. In FIG. 6H, the drawstring ends 12 and 14 have been cut near the stent 2. In FIG. 6I, the sheath introducer 302 has been removed from the patient. In FIG. 6J, the stent 2 is shown in the process of being absorbed at the implantation site as the vein collapses. In FIG. 6K, the absorption of the stent 2 is complete, the vein has fully collapsed, and a permanent blockage 320 remains.

It will be appreciated that other stent implantation methods may be used in accordance with the present invention. For example, instead of approaching the implantation site via the deep venous system, a superficial venous approach could be used by entering one of the long or short saphenous veins either above or below the implantation site, in relatively close proximity thereto. If such an entry point is used, an optional additional step that can be performed prior to cutting the drawstring ends 12 and 14 would be to suture one or both of them to the patient's skin at the entry point. This will help stabilize the stent 2 in its implantation position if such stabilization is desired. Other stabilization techniques could also be used, such as forming the stent 2 with a suitable surface-gripping configuration or with other gripping means.

It will also be appreciated that the use of a balloon catheter as per the exemplary method described above may not be necessary or desirable in all cases. For example, if a solid body stent, such as the stent 202 of FIGS. 5A and 5B is to be deployed, the use of a balloon catheter would not be indicated. In that case, the stent could be carried to the implantation site within an open-ended catheter (without a balloon dilator tip). A plunger can then be used to force the stent out of the catheter into engagement with the vein. Note that a stent might also be constructed with resilient properties so as to be outwardly expandable (e.g., using bio-absorbable foam). In that case, the stent would be compressed while in the catheter but would expand to engage the vein walls when deployed.

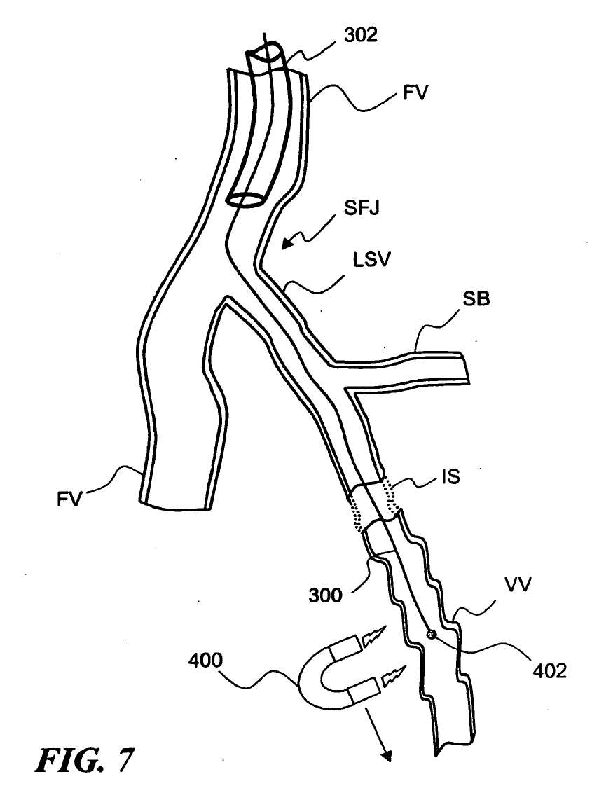

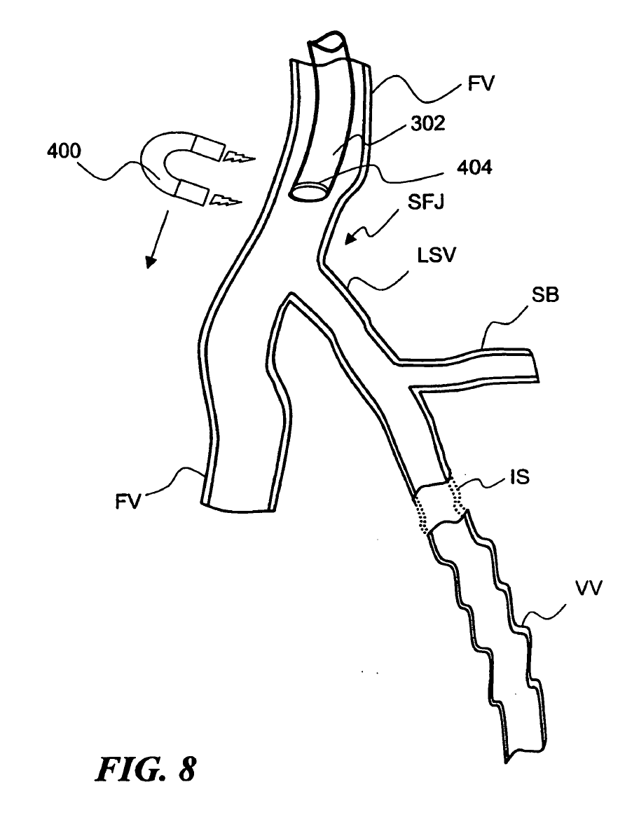

FIGS. 7 and 8 show a further aspect of the invention in which a magnet 400 is used to direct either the guide wire 300 or the sheath introducer 302 (without a guide wire) to the implantation site in the varicose vein (VV). In FIG. 7, the tip portion 402 of the guide wire 300 is made from ferromagnetic material. In FIG. 8, the tip of the sheath introducer 302 is provided with a ferromagnetic guide element 404. During treatment, a physician manipulates the magnet 400 over the surface of a patient's skin. Because of the proximity of the superficial venous system to the skin's surface, the magnet 400 will impart a magnetic force on the guide wire tip 402 or the introducer guide element 404, thereby pulling the tip or guide element in a direction determined by the magnet's movement. In this way, and with the possible assistance of conventional optical guidance means, the stent 2 can be deployed to the desired location.

Accordingly, an apparatus and method for treating varicose veins are disclosed. While various embodiments of the invention have been shown and described, it should be apparent that many variations and alternative embodiments could be implemented in accordance with the teachings herein. For example, although various bio-absorbable stent constructions have been described using bio-absorbable fabrics, filaments and foams, it will be appreciated that other bio-absorbable constructions may also be used for stents designed in accordance with the invention. Examples include solid surface materials that could be configured to form a stent using molding, milling or other fabrication techniques. It will be further appreciated that the stent need not necessarily be 100% efficient at blocking blood flow. It is sufficient that there be enough blood flow suppression to induce clotting and fibrosis at the implantation site. Relatedly, it is noted that not all portions of the stent need to block blood flow so long as the stent's closure portion fulfills that function. Thus, side portions of the stent that engage the venous wall could potentially be porous to blood flow so long as the stent's closure portion (e.g., an end wall) substantially blocks blood flow. It is understood, therefore, that the invention is not to be in any way limited except in accordance with the spirit of the appended claims and their equivalents.

1-20. (canceled)

21. A method of treating a hollow anatomical structure, said method comprising:
inserting into said hollow anatomical structure an implant formed from a bioabsorbable material;
providing a fluid flow path through said implant; and
causing blockage of said flow path after said inserting.

22. The method of claim 21, further comprising causing a post-implantation, substantially complete occlusion of said hollow anatomical structure with said implant.

23. The method of claim 21, wherein said hollow anatomical structure comprises a blood vessel.

24. The method of claim 21, wherein said hollow anatomical structure comprises a varicose vein.

25. The method of claim 21, further comprising permitting said implant to be absorbed into a patient in which said implant is inserted.

26. The method of claim 21, further comprising promoting blood clotting and fibrosis in said hollow anatomical structure near said implant.

27. The method of claim 26, further comprising permitting said implant to be absorbed as said blood clotting and fibrosis proceeds.

28. A method of treating a hollow anatomical structure of a patient, said method comprising:
implanting a bioabsorbable occluder into said hollow anatomical structure;
with said bioabsorbable occluder, causing a partial occlusion of said hollow anatomical structure; and
with said bioabsorbable occluder, causing a subsequent, substantially complete occlusion of said hollow anatomical structure.

29. The method of claim 28, further comprising fixing said bioabsorbable occluder in said hollow anatomical structure to inhibit post-implantation movement of said bioabsorbable occluder within said hollow anatomical structure.

30. The method of claim 29, wherein said bioabsorbable occluder comprises a fixation member, and fixing said bioabsorbable occluder comprises securing said fixation member with respect to the anatomy of said patient.

31. The method of claim 30, wherein fixing said bioabsorbable occluder comprises securing said fixation member with respect to the anatomy of said patient at a fixation location which is spaced longitudinally along said hollow anatomical structure from an end of said bioabsorbable occluder.

32. The method of claim 30, wherein fixing said bioabsorbable occluder comprises securing said fixation member with respect to the anatomy of said patient at a fixation location spaced from said bioabsorbable occluder.

33. The method of claim 28, wherein said bioabsorbable occluder has a proximal end and a distal end spaced longitudinally from said proximal end, said bioabsorbable occluder has an occluder body extending from said proximal end to said distal end, said occluder body has an outer side portion configured to engage an inner wall of said hollow anatomical structure, and said occluder body is bioabsorbable.

34. The method of claim 28, wherein said hollow anatomical structure comprises a blood vessel.

35. The method of claim 28, wherein said hollow anatomical structure comprises a varicose vein.

36. The method of claim 28, further comprising permitting said occluder to be absorbed into a patient in which said occluder is implanted.

37. The method of claim 28, further comprising promoting blood clotting and fibrosis in said hollow anatomical structure near said occluder.

38. The method of claim 37, further comprising permitting said occluder to be absorbed as said blood clotting and fibrosis proceeds.

39. A method of reducing flow through a blood vessel, said method comprising:
inserting into said blood vessel an occluder comprising a bioabsorbable material;
fixing said occluder in said hollow anatomical structure to inhibit post-implantation movement of said bioabsorbable occluder within said hollow anatomical structure;
with said occluder, causing a partial occlusion of said blood vessel; and
with said occluder, causing a subsequent, substantially complete occlusion of said blood vessel.

40. The method of claim 39, further comprising substantially spanning a lumen of said blood vessel with said occluder.

41. The method of claim 39, further comprising permitting said occluder to be absorbed into the body of a patient.

42. The method of claim 39, wherein said blood vessel comprises a varicose vein.

43. Apparatus comprising:
an implant formed from a bioabsorbable material, said implant having a distal end and a proximal end opposite said distal end;
said implant having a first state in which a fluid flow path passes through said implant between said distal end and said proximal end, and a second state in which substantially no fluid flow path passes through said implant.

44. The apparatus of claim 43, wherein no fluid flow path passes through said implant when said implant is in said second state.

45. The apparatus of claim 43, wherein:
said distal end of said implant is spaced longitudinally from said proximal end; and
said implant further comprises a stabilization member which extends longitudinally from one end of said implant.

46. The apparatus of claim 45, wherein said stabilization member comprises an elongate bioabsorbable member.

47. The apparatus of claim 43, wherein said implant is formed entirely from bioabsorbable material.
